(19) United States
(12) Patent Application Publication
(10) Pub. No.: US 2026/0239876 A1
(43) Pub. Date: Aug. 13, 2026

(54) BATHOCUPROINE DERIVATIVE, AND OPTOELECTRONIC DEVICE HAVING BUFFER LAYER CONTAINING SAME

(71) Applicants: Pusan National University Industry-University Cooperation Foundation, Gyeongsangnam-do (KR); IUCF-HYU (Industry-University Cooperation Foundation Hanyang University), Seoul (KR)

(72) Inventors: Ji Youn SEO, Busan (KR); In Hwan JUNG, Seoul (KR); Do Yeong YEO, Seoul (KR)

(73) Assignees: Pusan National University Industry-University Cooperation Foundation, Gyeongsangnam-do (KR); IUCF-HYU (Industry-University Cooperation Foundation Hanyang University), Seoul (KR)

(21) Appl. No.: 19/387,563

(22) Filed: Nov. 12, 2025

(30) Foreign Application Priority Data
Feb. 11, 2025 (KR) 10-2025-0017549

(51) Int. Cl. H10K 85/60; H10K 30/50; H10K 30/82; H10K 30/85; H10K 85/50

(52) U.S. Cl. CPC H10K 85/6572; H10K 30/50; H10K 30/82; H10K 30/85; H10K 85/50

(57) ABSTRACT

Proposed are a bathocuproine (BCP) derivative with improved molecular planarity, and an optoelectronic device including the BCP derivative as a buffer layer thus having enhanced long-term stability. More particularly, proposed is a bathocuproine (BCP) derivative, wherein a 1,10-phenanthroline compound is unsubstituted at 4- and 7-positions, and is symmetrically substituted at 2- and 9-positions with phenyl groups, alkylphenyl groups, halophenyl groups thereby providing improved molecular planarity. According to the present disclosure, it is possible to employ the BCP derivative as a novel tailor-made buffer material for an electron transport layer, which overcomes the stereochemical limitations of BCP as a conventional buffer layer material while also exhibiting excellent electrical properties.

### CROSS-REFERENCE TO RELATED APPLICATION

The present application claims priority to Korean Patent Application No. 10-2025-0017549, filed Feb. 11, 2025, the entire contents of which is incorporated herein for all purposes by this reference.

### BACKGROUND OF THE INVENTION

### Field of the Invention

The present disclosure relates generally to a bathocuproine (BCP) derivative, and optoelectronic device having a buffer layer containing the same. More particularly, the present disclosure relates to a bathocuproine (BCP) derivative with improved molecular planarity, and a perovskite solar cell including the BCP derivative as a buffer layer thus having enhanced long-term stability.

### Description of the Related Art

Optoelectronic devices operate based on the principle that light induces physical phenomena such as the generation, recombination, and transport of electrons and holes within a semiconductor. These devices are generally classified into two categories: those that convert light into electrical signals, such as photodetectors and solar cells, and those that convert electrical signals into light, such as light-emitting diodes (LEDs) and laser diodes. Representative examples include LEDs, laser diodes, photodiodes, solar cells, image sensors, and fiber-optic transmitters and receivers. Such devices are widely applied across various industrial fields, including displays, lighting, optical communications, sensors, medical instruments, industrial measurement and automation, and energy conversion systems such as solar power generation. Recently, research has increasingly focused on devices utilizing novel materials such as two-dimensional semiconductors and perovskites, enabling high performance, high integration, and ultra-high-speed signal processing.

In particular, with growing concerns about climate change, solar cells are receiving increased attention as a key renewable energy source. Solar cells are devices that convert sunlight into electrical energy through the photovoltaic effect. Among them, perovskite solar cells are a type of solar cell that utilizes a material with a perovskite structure as a photoactive layer. They are considered a promising next-generation solar cell due to their lightweight design and simple manufacturing processes and thus have been the subject of various studies. Perovskite solar cells (PSCs) have demonstrated power conversion efficiencies exceeding 26% for single-junction devices, and research efforts are ongoing to pave the way for their commercialization.

In particular, interface contact in PSCs is a critical factor significantly influencing the overall performance and efficiency of the device. These interfaces, which exist between different layers within the PSC structure, exert a significant effect on charge carrier transport, recombination, and extraction, which are all vital processes for the conversion of sunlight into electricity.

A perovskite layer, which acts as an active layer, initiates the generation of electron-hole pairs. These charge carriers then migrate through an electron transport layer (ETL) and a hole transport layer (HTL) to ultimately reach electrodes. However, the formation of a Schottky barrier at the interface between the electron transport layer (ETL) and the electrode can impede free movement of electrons. This requires the introduction of a buffer layer such as Tris(8-hydroxyquinolinato) aluminum (Alq₃), Tin(IV) oxide (SnO₂), and bathocuproine (BCP). Of these, BCP is gaining popularity in PSCs due to its ease of application through solution coating or thermal evaporation and its ability to provide high performance. Therefore, BCP is often used as a buffer layer between the ETL layer and the electrode, playing a crucial role in facilitating ohmic contact and reducing interfacial charge recombination. It also acts as a barrier, preventing the inter-diffusion of harmful species into a perovskite absorber while suppressing the out-diffusion of decomposition products.

However, despite the excellent electrical properties of BCP, BCP has a limitation that it is readily reacts with moisture and oxygen, leading to degradation of device long-term stability when exposed to air during manufacturing or usage.

Accordingly, the inventors of the present disclosure have developed a BCP derivative as a novel tailor-made buffer material for an electron transport layer, which overcomes the stereochemical limitations of conventional BCP while also exhibiting excellent electrical properties, thereby completing the present disclosure.

The foregoing is intended merely to aid in the understanding of the background of the present disclosure, and is not intended to mean that the present disclosure falls within the purview of the related art that is already known to those skilled in the art.

### DOCUMENTS OF RELATED ART

(Non-Patent document 1) Impact of Solution-Processed BCP Buffer Layer on Efficient Perovskite Solar Cells (Journal of the Korean institute of electronic material engineers, v. 34 no. 1, 2021, pp. 73-77)

### SUMMARY OF THE INVENTION

Accordingly, the present disclosure has been made keeping in mind the above problems occurring in the related art, and one objective of the present disclosure is to provide a bathocuproine (BCP) derivative.

Another objective of the present disclosure is to provide an optoelectronic device having a buffer layer containing the BCP derivative.

Still another objective of the present disclosure is to provide a perovskite solar cell having a buffer layer containing the BCP derivative and a method for manufacturing the same.

In order to achieve the above objectives, according to one aspect of the present disclosure, there is provided a bathocuproine (BCP) derivative, wherein a 1,10-phenanthroline compound is unsubstituted at 4- and 7-positions, and is symmetrically substituted at 2- and 9-positions with phenyl groups, alkylphenyl groups, halophenyl groups thereby providing improved molecular planarity.

In the present disclosure, the derivative may have Chemical Formula 1 below:

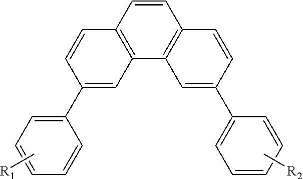

(where R₁, R₂ are each independently selected from the group consisting of hydrogen, an alkyl group having 1 to 4 carbon atoms, and a halogen atom)

According to another aspect of the present disclosure, there is provided a perovskite solar cell, wherein a first electrode, a hole transport layer, a photoactive layer, an electron transport layer, and a second electrode may be sequentially stacked, and wherein the photoactive layer may be formed to include a perovskite compound represented by the chemical formula APbX₃ or ASnX₃ (where A is a metal cation, an organic cation, or a combination thereof, and X is selected from at least one of I, Br, and Cl), the electron transport layer may be formed to include a fullerene compound, and an electron transport buffer layer containing the BCP derivative may be further provided between the electron transport layer and the second electrode.

In the present disclosure, the first electrode may include at least one selected from the group consisting of a flexible transparent electrode substrate, indium tin oxide (ITO), fluorine tin oxide (FTO), aluminum zinc oxide (AZO), boron-doped zinc oxide (BZO), niobium titanium oxide (NTO), zinc tin oxide (ZTO), and indium zinc tin oxide (IZTO), and the second electrode may include at least one selected from the group consisting of silver (Ag), aluminum (Al), platinum (Pt), tungsten (W), copper (Cu), molybdenum (Mo), gold (Au), nickel (Ni), palladium (Pd), and carbon (C).

Additionally, in present disclosure, the perovskite solar cell may have a power conversion efficiency of at least 90% after 10 hours of operation under room temperature and low humidity conditions (20 to 25% RH).

Additionally, in present disclosure, the perovskite solar cell may have a power conversion efficiency of at least 50% after 50 hours of operation under room temperature and high humidity conditions (80 to 85% RH).

According to still another aspect of the present disclosure, there is provided a method of manufacturing a perovskite solar cell, the method including: forming a first electrode; forming a hole transport layer on the first electrode; forming a photoactive layer by coating a perovskite compound of APbX₃ or ASnX₃ (where A is a metal cation, an organic cation, or a combination thereof, and X is at least one selected from I, Br, and Cl) on the hole transport layer, followed by heat treatment; forming an electron transport layer by depositing a fullerene compound on the photoactive layer; forming a buffer layer by depositing the BCP derivative on the electron transport layer; and forming a second electrode on the buffer layer.

According to the present disclosure, it is possible to provide a BCP derivative as a novel tailor-made buffer material for an electron transport layer, which overcomes the stereochemical limitations of BCP as a conventional solar cell buffer layer material while also exhibiting excellent electrical properties.

The BCP derivative of the present disclosure is characterized in that a 1,10-phenanthroline compound is unsubstituted at 4- and 7-positions, and is symmetrically substituted at 2- and 9-positions with phenyl groups, alkylphenyl groups, or halophenyl groups thereby providing improved molecular planarity. The improved molecular planarity allows for the most effective protection of reactive nitrogen atoms in a phenanthroline core, contributing to enhanced charge transport of PSC, reduced hysteresis, improved device performance, and improved device stability.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 illustrates a schematic diagram of synthetic routes of BCP-m1 and BCP-m2 according to an embodiment of the present disclosure;

FIGS. 2a to 2d illustrate ¹H nuclear magnetic resonance (NMR) and ¹³C NMR graphs of BCP-m1 and BCP-m2 according to an embodiment of the present disclosure;

FIG. 3 illustrates the 2D and 3D chemical structures of BCP, BCP-m1, and BCP-m2 according to an embodiment of the present disclosure;

FIG. 4 illustrates the optimized geometrical structures of BCP-C₆₀, BCP-m1-C₆₀, and BCP-m2-C₆₀ complexes according to an embodiment of the present disclosure;

FIGS. 5a and 5b illustrate absorption spectra in solution and film states according to an embodiment of the present disclosure;

FIG. 6 illustrates simulated electrostatic potential maps of (a-c) BCP, BCP-m1, and BCP-m2, and (d-f) interactions between C₆₀ and BCP, BCP-m1, and BCP-m2 according to an embodiment of the present disclosure;

FIGS. 7a to 7d illustrate solutions in which BCP, BCP-m1, and BCP-m2 are each dissolved under the same conditions, and surface roughness (AFM) measurement results of thin films coated with each solution according to an embodiment of the present disclosure;

FIG. 8 is a schematic view illustrating a perovskite solar cell manufacturing process according to an embodiment of the present disclosure;

FIG. 9 illustrates AFM images of (a) BCP, (c) BCP-m1, and (e) BCP-m2, and line profile graphs obtained from the AFM images of (b) BCP, (d) BCP-m1, and (f) BCP-m2 according to an embodiment of the present disclosure;

FIG. 10 illustrates EFM surface electrostatic force images of (a) BCP, (c) BCP-m1, and (e) BCP-m2, and line profile graphs obtained from the EFM images of (b) BCP, (d) BCP-m1, and (f) BCP-m2 according to an embodiment of the present disclosure;

FIG. 11 illustrates graphs of (a) steady-state PL spectra and (b) TRPL measurement results of perovskite with and without a top coating layer according to an embodiment of the present disclosure;

FIGS. 12a to 12i illustrate (a) a schematic structure of a perovskite solar cell according to an embodiment of the present disclosure, (b) a schematic diagram of bandgap alignment of BCPs, (c) a schematic band diagram of BCP, BCP-m1, and BCP-m2, a box chart showing statistical distribution of (d) current density (J_{SC}), (e) open-circuit voltage (V_{OC}), (f) fill factor (FF), and (g) power conversion efficiency (PCE) under simulated AM 1.5G sunlight, (h) a series resistance impedance spectroscopy graph of PSCs with BCP, BCP-m1, and BCP-m2, and (i) a graph of J-V curves in reverse scans (solid line) and forward scans (dash line) for perovskite solar cells under various conditions;

FIG. 13 illustrates graphs of (a) selective contact resistance and (b) recombination resistance by impedance spectroscopy of a PSC using BCP, BCP-m1, and BCP-m2 according to an embodiment of the present disclosure; and

FIG. 14 illustrates MPPT graphs of a perovskite solar cell under light soaking (AM 1.5 G, 100 Mw cm⁻²) (a) in 20 to 25% humidity at room temperature with encapsulation and (b) in 80 to 85% humidity at room temperature without encapsulation according to an embodiment of the present disclosure.

### DETAILED DESCRIPTION OF THE INVENTION

The present disclosure will be described in detail below.

Throughout the specification, when a certain portion “includes” or “comprises” a certain component, this indicates that other components are not excluded and may be further included unless otherwise noted.

In one aspect, the present disclosure relates to a bathocuproine (BCP) derivative, characterized in that a 1,10-phenanthroline compound is unsubstituted at 4- and 7-positions, and is symmetrically substituted at 2- and 9-positions with phenyl groups, alkylphenyl groups, or halophenyl groups thereby providing improved molecular planarity.

Preferably, the derivative is characterized by having Chemical Formula 1 below:

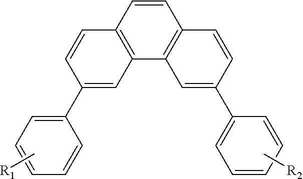

(where R₁, R₂ are each independently selected from the group consisting of hydrogen, an alkyl group having 1 to 4 carbon atoms, and a halogen atom)

The BCP derivative of the present disclosure is characterized by reducing a dihedral angle between a central phenanthroline unit and phenyl rings symmetrically attached at the 2- and 9-positions, thereby enhancing molecular planarity. The enhanced molecular planarity allows for the most effective protection of reactive nitrogen atoms in a phenanthroline core, contributing to enhanced charge transport of an optoelectronic device, reduced hysteresis, improved device performance, and improved device stability.

Therefore, in another aspect, the present disclosure provides an optoelectronic device, wherein a first electrode, a hole transport layer, a photoactive layer, an electron transport layer, and a second electrode are sequentially stacked, and an electron transport buffer layer containing the BCP derivative is further provided between the electron transport layer and the second electrode. The optoelectronic devices may be, for example, LEDs, laser diodes, photodiodes, solar cells, image sensors, and fiber-optic transmitters and receivers.

In addition, the derivative of the present disclosure can significantly reduce steric hindrance when applied to a solar cell due to the enhancement of molecular planarity. This reduction in steric hindrance leads to closer packing of molecules and enhanced intramolecular interactions and charge transport, ultimately increasing efficiency and long-term stability of the solar cell.

Therefore, in another aspect, the present disclosure provides a perovskite solar cell, wherein a first electrode, a hole transport layer, a photoactive layer, an electron transport layer, and a second electrode are sequentially stacked, and wherein the photoactive layer is formed to include a perovskite compound represented by the chemical formula APbX₃ or ASnX₃ (where A is a metal cation, an organic cation, or a combination thereof, and X is selected from at least one of I, Br, and Cl), the electron transport layer is formed to include a fullerene compound, and an electron transport buffer layer containing the BCP derivative is further provided between the electron transport layer and the second electrode.

In the present disclosure, the electron transport buffer layer reduces steric hindrance and enhances molecular planarity by introducing phenyl groups at the 2- and 9-positions of the phenanthroline core, thereby enabling efficient π-conjugation and π-π intermolecular stacking, thus most effectively suppressing the reactivity of phenanthroline in the solar cell and increasing device stability. According to a preferred embodiment of the present disclosure, the perovskite solar cell may have a power conversion efficiency of at least 90% after 10 hours of operation under room temperature and low humidity conditions (20 to 25% RH).

Additionally, according to a preferred embodiment of the present disclosure, a solar cell having a BCP derivative buffer layer of the present disclosure was found to exhibit a lifespan more than twice that of a conventional BCP buffer layer. That is, the electron transport buffer layer of the present disclosure can effectively enhance long-term stability of the solar cell by structurally maintaining stability and protecting it from moisture even in high-humidity external environments. Therefore, preferably, the perovskite solar cell of the present disclosure may have a power conversion efficiency of at least 50% after 50 hours of operation under room temperature and high humidity conditions (80 to 85% RH).

Also preferably, the electron transport buffer layer may have an average surface roughness of up to 10 nm. When the average surface roughness of the electron transport buffer layer exceeds 10 nm, there may be a problem of decreased charge transport efficiency.

Also preferably, the electron transport buffer layer may have a thickness of 2 to 10 nm. When the thickness of the electron transport buffer layer is less than 2 nm, there may be a problem of reduced coverage, whereas when it exceeds 10 nm, there may be a problem of decreased charge transport efficiency.

In one embodiment, the perovskite represented by ABX₃ may be a compound, where A is an alkali metal, an organic cation (e.g., organic ammonium) and/or an inorganic cation, B is a metal material, and X is selected from a halogen anion, a chalcogenide anion, and SCN-(thiocyanate). For example, A may be an alkali metal such as Na, K, Rb, Cs, or Fr; (CH₃NH₃)ₙ, ((CₓH₂ₓ₊₁)ₙNH₃)₂(CH₃NH₃)ₙ, (RNH₃)₂, (CₙH₂ₙ₊₁NH₃)₂, (CF₃NH₃), (CF₃NH₃)ₙ, ((CₓF₂ₓ₊₁)ₙNH₃)₂(CF₃NH₃)ₙ, ((CₓF₂ₓ₊₁)ₙNH₃)₂, or (CₙF₂ₙ₊₁NH₃)₂ (where n is an integer of equal to or greater than 1, and x is an integer of equal to or greater than 1); B may be a divalent transition metal, a rare earth metal, an alkaline earth metal, Pb, Sn, Ge, Ga, In, Al, Sb, Bi, or Po; and X may be P, Cl, Br, or I. In some examples, the perovskite may be CH₃NH₃PbI₃, CH₃NH₃PbBr₃, CH₃NH₃PbI₂Cl, or CH₃NH₃PbI₂Br. In some examples, when two or more of the above perovskite compounds are included, the ratio of a first perovskite compound to a second perovskite compound may be 5:5 to 9:1. In some examples, the perovskite may further include compounds represented by the chemical formulas NaX′, ZnX′, KX′, and CsX′ (where X is selected from Cl, Br, and I), which may be included in an amount of equal to or less than 10% or equal to or less than 5%, based on the total perovskite compound.

The photoactive layer severs to separate electrons and holes towards two electrodes and direct electrons and holes towards an electron transport layer and a hole transport layer, respectively.

Perovskite grains in the photoactive layer may have a size of 1 to 900 nm, 1 to 800 nm, 5 to 600 nm, 5 to 300 nm, 10 to 300 nm, 10 to 100 nm, or 10 to 50 nm.

The photoactive layer may have a thickness of 500 to 800 nm or 550 to 700 nm, preferably, 550 to 650 nm to improve the performance of the photoactive layer.

Also preferably, the first electrode may include at least one selected from the group consisting of a flexible transparent electrode substrate, indium tin oxide (ITO), fluorine tin oxide (FTO), aluminum zinc oxide (AZO), boron-doped zinc oxide (BZO), niobium titanium oxide (NTO), zinc tin oxide (ZTO), and indium zinc tin oxide (IZTO), and the second electrode may include at least one selected from the group consisting of silver (Ag), aluminum (Al), platinum (Pt), tungsten (W), copper (Cu), molybdenum (Mo), gold (Au), nickel (Ni), palladium (Pd), and carbon (C).

The solar cell of the present disclosure may be manufactured by sequentially stacking the first electrode, the hole transport layer, the photoactive layer, the electron transport layer, and the second electrode. In another aspect, the present disclosure provides a method of manufacturing a perovskite solar cell, characterized by including the steps of: forming a first electrode; forming a hole transport layer on the first electrode; forming a photoactive layer by coating a perovskite compound of APbX₃ or ASnX₃ (where A is a metal cation, an organic cation, or a combination thereof, and X is at least one selected from I, Br, and Cl) on the hole transport layer, followed by heat treatment; forming an electron transport layer by depositing a fullerene compound on the photoactive layer; forming a buffer layer by depositing the BCP derivative on the electron transport layer; and forming a second electrode on the buffer layer.

In one embodiment, in forming the first electrode, the first electrode as mentioned in the present disclosure may be prepared, and for example, a substrate having a conductive material layer coated thereon may be used or a conductive layer may be formed on a substrate using sputtering, chemical vapor deposition (CVD), deposition, solution processes, coating, printing, or the like.

Additionally, in forming the hole transport layer, the hole transport layer may be formed using sputtering, chemical vapor deposition (CVD), deposition, solution processes, coating, printing, or the like, but is not limited thereto.

Additionally, in forming the photoactive layer, a coating or deposition process may be employed. For example, a perovskite layer may be formed by at least one method selected from spin coating, spray coating, slot die coating, blade coating, and deposition. In one example, the deposition may be performed by at least one method selected from chemical vapor deposition (CVD), atomic layer deposition (ALD), plasma-enhanced atomic layer deposition (PEALD), molecular beam epitaxy deposition, and physical vapor deposition, but is not limited thereto.

Additionally, in forming the electron transport layer, a coating process may be used. The coating process may be performed by at least one method selected from spin coating, spray coating, slot die coating, and blade coating, but is not limited thereto.

Additionally, in forming the electron transport buffer layer, the electron transport buffer layer may be formed using sputtering, chemical vapor deposition (CVD), deposition, solution processes, coating, printing, or the like, but is not limited thereto.

Additionally, in forming the second electrode, an electrode may be formed by a high-temperature process. For example, the electrode may be formed by a sputtering process under at least one of the following process conditions: 10 to 200 W, a temperature of 25° C. to 100° C., and an oxygen flow rate of 0 to 100 sccm.

At this time, the sputtering may be performed by ion-beam sputtering, reactive sputtering, ion-assisted deposition, high-target-utilization sputtering (HiTUS), high-power impulse magnetron sputtering (HiPIMS), gas flow sputtering, plasma sputtering, or the like, and the deposition may be performed by chemical vapor deposition (CVD), thermal evaporation, plasma sputtering, e-beam evaporation, atomic layer deposition (ALD), or the like. The coating may be performed by a solution process, and for example, may refer to a film-forming process using a liquid solvent, such as spin coating, spray coating, dip coating, inkjet printing, roll-to-roll printing, or screen printing.

Hereinafter, the present disclosure will be described in more detail with reference to examples, but the present disclosure is not limited thereto.

### Example

### Materials

1,1′-Bis(diphenylphosphino)ferrocene]dichloropalladium(II) was purchased from Sigma-Aldrich, and 2-Phenyl-4,4,5,5-tetramethyl-1,3,2-dioxaborolane and 4,4,5,5-Tetramethyl-2-(p-tolyl)-1,3,2-dioxaborolane were purchased from TCI Chemical. 1,4-dioxane and NaOH were purchased from Daejung, and 2,9-dichloro-1,10-phenanthroline was purchased from J Science (Korea). Methylammonium bromide (CH₃NH₃Br) and formamidinium iodide (CH(NH₂)₂I) were purchased from Greatcell Solar. Cesium iodide (CsI), lead(II) iodide (PbI₂), lead(II) bromide (PbBr₂), N,N-dimethylformamide (DMF), dimethyl sulfoxide (DMSO), MeO-4PACz, C₆₀, bathocuproine (BCP), ethyl acetate (EA), and methanol (CH₃OH) were used, and all these chemicals were employed without further purification.

### Synthesis of BCP-m1 and BCP-m2

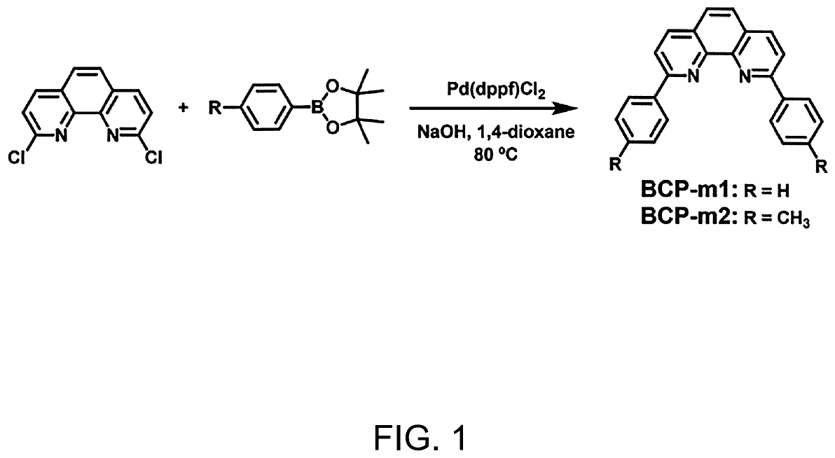

The synthesis process is illustrated in FIG. 1. Specifically, 2,9-dichloro-1,10-phenanthroline (500 mg, 2 mmol), 2-phenyl-4,4,5,5-tetramethyl-1,3,2-dioxaborolane (900 mg, 4.4 mmol) and [1,1′-bis(diphenylphosphino)ferrocene]dichloropalladium(II) (22 mg, 0.03 mmol) were dissolved in 1,4-dioxane under a nitrogen atmosphere and stirred at 80° C. Subsequently, an aqueous NaOH solution (530 g, 13.2 mmol, 6.6 ml) was slowly added to the reaction mixture and refluxed at 80° C. for 16 hours. The mixture was extracted with CH₂Cl₂ and washed several times with H₂O and brine. The combined organic layers were dried over anhydrous MgSO₄, filtered, and evaporated under reduced pressure. The crude product was purified by column chromatography on silica gel using ethyl acetate/hexane (v/v, 1/4) to yield BCP-m1 as a white solid (320 mg, 48.5%).

Except that 4,4,5,5-tetramethyl-2-(p-tolyl)-1,3,2-dioxaborolane (960 mg, 4.4 mmol) was used instead of 2-phenyl-4,4,5,5-tetramethyl-1,3,2-dioxaborolane (900 mg, 4.4 mmol), the same synthesis procedure as that of BCP-m1 was conducted, thereby yielding BCP-m2 as a white solid (640 mg, 85.3%).

The synthesis process is illustrated in FIG. 1. Furthermore, density functional theory (DFT) calculations were performed using the Gaussian 16 software package to elucidate the electronic structures of BCP, BCP-m1, and BCP-m2 buffer materials and their interactions with C₆₀.

The characteristics of the synthesized BCP-m1 and BCP-m2 were analyzed, and the results are illustrated in in FIGS. 1 to 7d.

First, FIG. 1 illustrates the synthesis process of BCP derivatives according to the example. Noble BCP derivatives, BCP-m1 and BCP-m2, were synthesized via a one-step Suzuki coupling reaction of 2,9-dichloro-1,10-phenanthroline with phenyl and p-tolyl boronic esters, respectively. The synthesized BCP-m1 and BCP-m2 were obtained as white crystalline solids with reaction yields of 50% and 85%, respectively, and were highly soluble and air-stable.

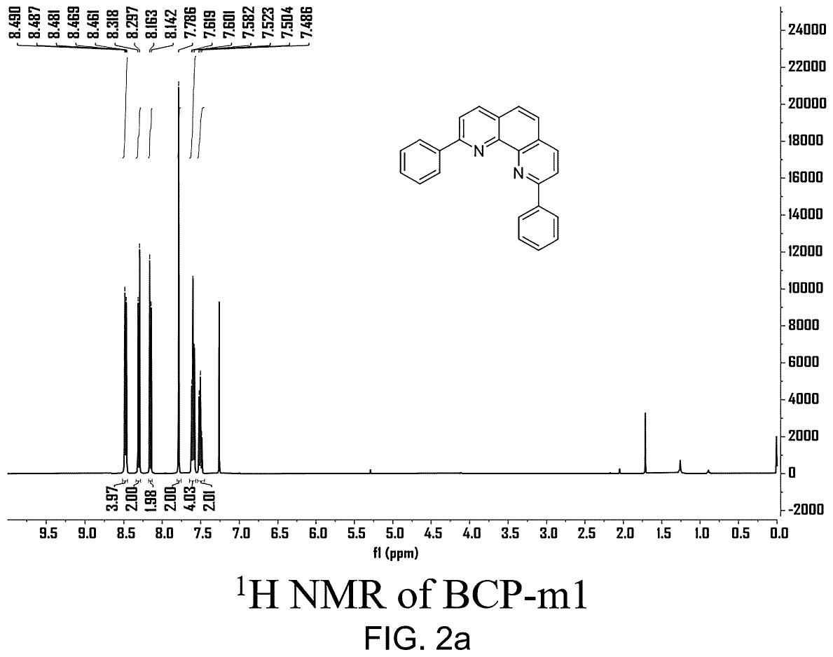

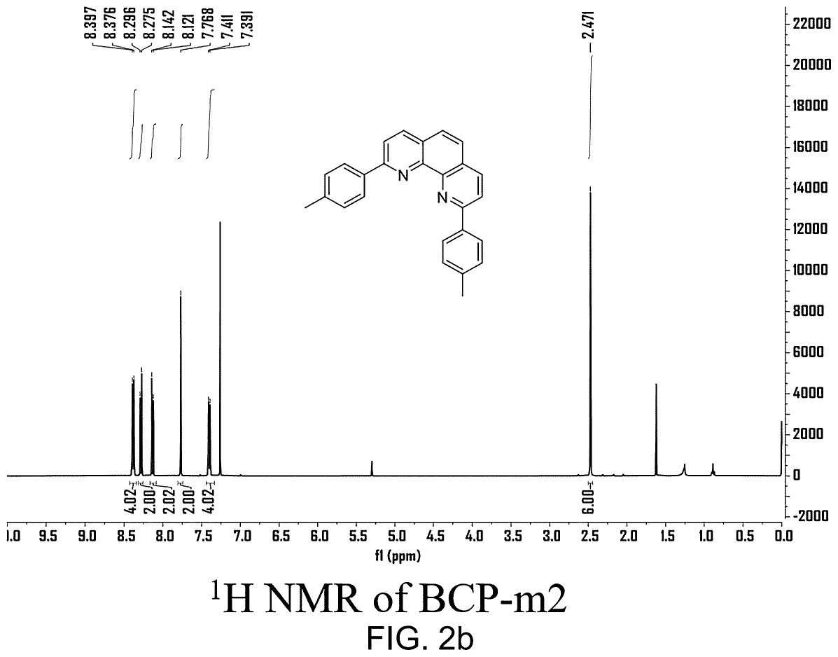

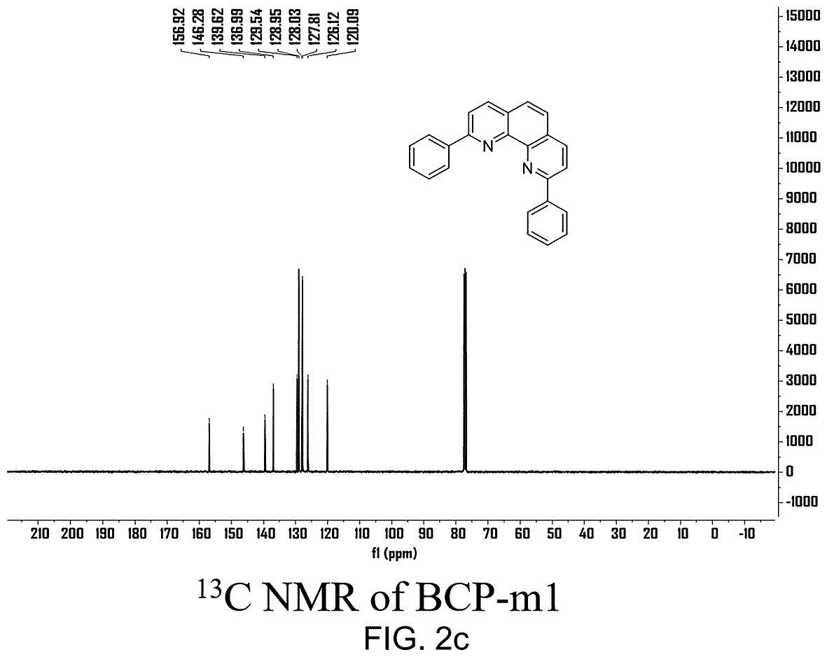

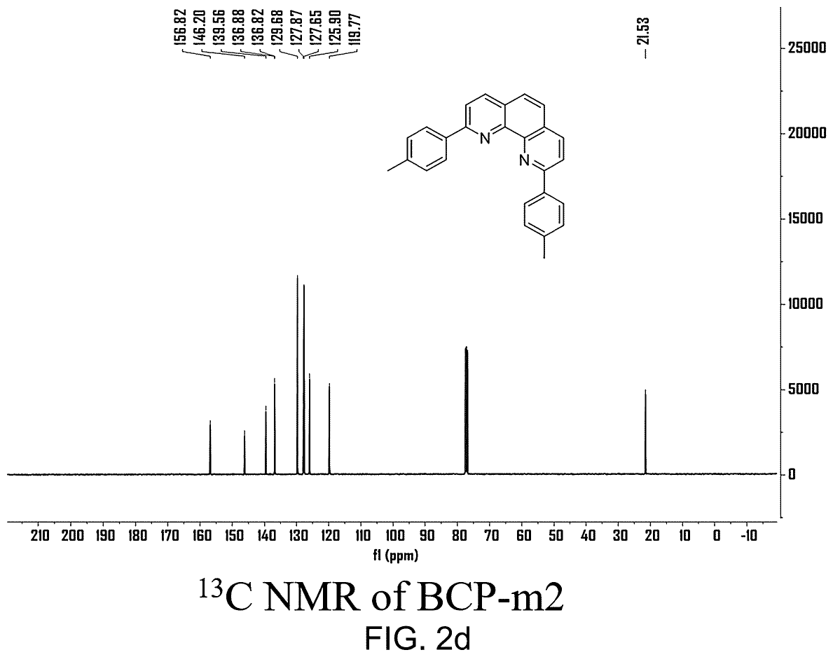

FIGS. 2a to 2d illustrate ¹H nuclear magnetic resonance (NMR) and ¹³C NMR graphs of BCP-m1 and BCP-m2, showing the successful synthesis of BCP-m1 and BCP-m2. Furthermore, BCP-m1 and BCP-m2 with molecular weights of 332 g mol⁻¹ and 360 g mol⁻¹, respectively, were designed to maintain a similar molecular weight to the BCP molecule (360 g mol⁻¹). Since the molecular weights of the synthesized buffer molecules are nearly identical to that of the BCP, the conventional vacuum deposition process can be used without significant modifications.

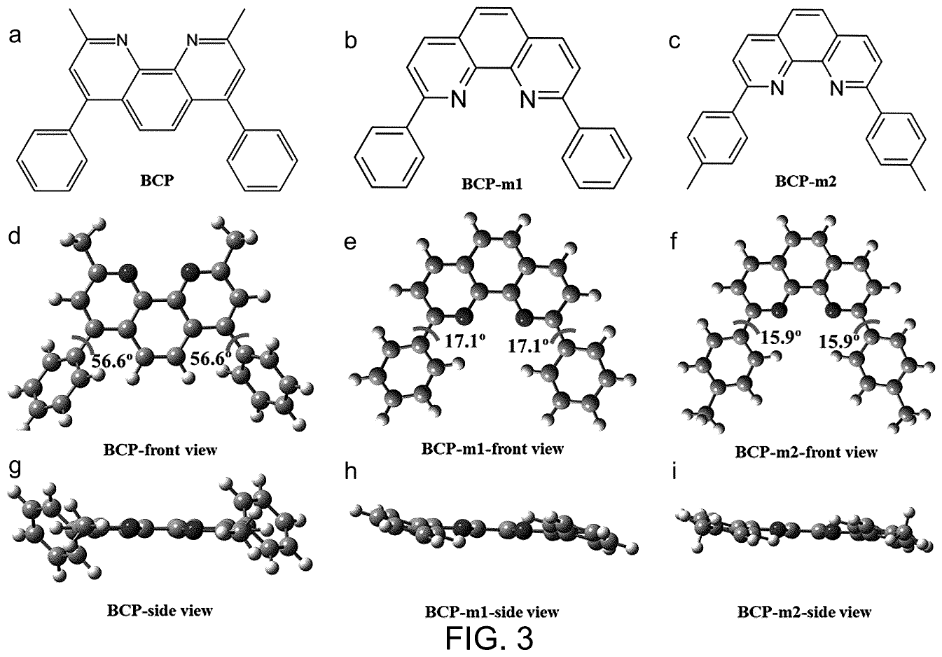

Additionally, FIG. 3 illustrates the results of simulating the geometrical structure and electron density distribution of BCP, BCP-m1, and BCP-m2 through DFT calculations, illustrating (a-c) the 2D chemical structures of BCP, BCP-m1, and BCP-m2, and (d-f) front views and (g-i) side views of DFT-optimized 3D structures (where hydrogen, carbon, and nitrogen atoms are represented by white, gray, and blue spheres, respectively).

Referring to this, d and g of FIG. 3 show that the steric hindrance caused by methyl groups can limit close packing of molecules, potentially reducing the efficiency of intermolecular interactions and charge transport. This indicates that under high thermal stress, methyl groups may also contribute to material degradation, which can result in the above problem in environments where solar cells are exposed to high temperatures for extended periods.

On the other hand, in the case of BCP-m1 and BCP-m2 synthesized in the above example, BCP-m1 was synthesized by replacing methyl groups by moving phenyl groups from 4- and 7-positions to 2- and 9-positions, and BCP-m2 was synthesized by replacing phenyl groups with tolyl groups.

In this regard, referring to d to f of FIG. 3, BCP exhibited a dihedral angle of 56.6°, while BCP-m1 and BCP-m2 derivatives exhibited significantly reduced dihedral angles of 17.1° and 15.9°, respectively. That is, the major structural modification implemented in BCP-m1 and BCP-m2 reduces a dihedral angle between a central phenanthroline unit and attached phenyl rings, thereby enhancing molecular planarity. In this regard, referring to h and i of FIG. 3, the 3D lateral structures of BCP-m1 and BCP-m2 show that the synthesized derivatives significantly reduce steric hindrance, thereby improving close packing of molecules and enhancing the efficiency of intramolecular interactions and charge transport. That is, enhanced planarity of the molecular skeleton can promote enhanced orbital overlap and charge transfer within materials.

As such, the phenyl ring on the control BCP was highly twisted from the BCP core unit, while BCP-m1 and BCP-m2 exhibited a fairly planar backbone structure. This implies that BCP-m1 and BCP-m2 can have efficient π-conjugation and π-π intermolecular stacking.

To investigate the impact of molecular modification on charge transport, the electron reorganization energies (EREs) of BCP, BCP-m1, and BCP-m2 were calculated. As a result, BCP-m1 and BCP-m2 showed significantly lower EREs of 0.256 eV and 0.245 eV, respectively, compared to BCP (0.425 eV). A low ERE is generally correlated with enhanced charge carrier mobility, suggesting that the structural modifications in BCP-m1 and BCP-m2 enhanced their ability to transport electrons. Binding energy (BE) calculations were performed to assess the interaction strength between C₆₀ and three BCP derivatives, as expressed by Equation 1 below.

$$BE = {E}_{Complex} - ( {E}_{BCP} + {E}_{C 60} ) \qquad [ Equation 1 ]$$

BE is a key parameter in understanding how strongly these buffer layer materials interact with C₆₀, which is essential for optimizing the performance of PSCs. The BE of a BCP derivative with C₆₀ was determined by subtracting the sum of the energies of the isolated BCP derivative and C₆₀ from the energy of a complex. The calculated BEs for C₆₀ complexes with BCP, BCP-m1, and BCP-m2 were determined to be −10.34 kcal/mol, −11.74 kcal/mol, and −14.20 kcal/mol, respectively. That is, a strong correlation was observed between increased molecular planarity and enhanced BE, indicating that molecular planarity plays a crucial role in strengthening the interaction between a buffer layer and an electron transport material. The reduced dihedral angle and increased BE in BCP-m1 and BCP-m2 facilitate better π-π stacking and more efficient electronic binding with C₆₀. This improvement in BE indicates the enhanced performance potential of these modified buffer layers in PSC applications where the stronger intermolecular interactions contribute to device efficiency and stability.

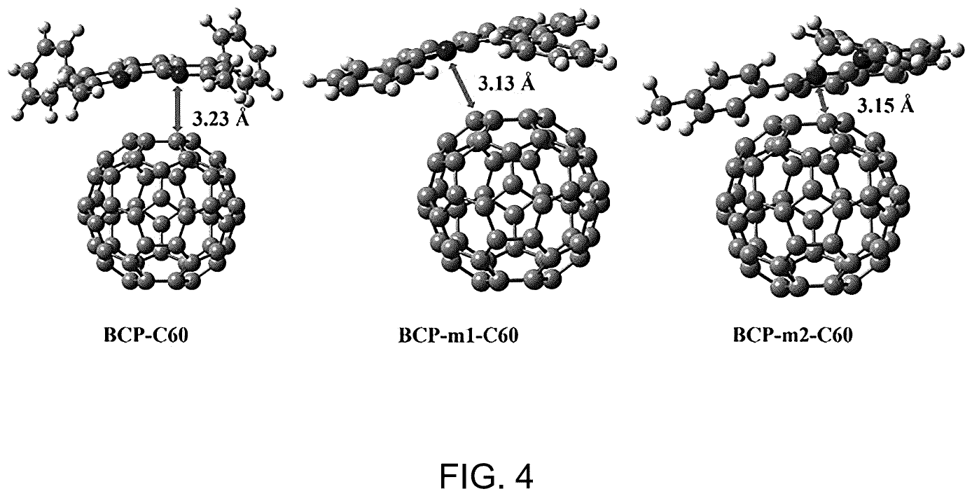

In this regard, FIG. 4 illustrates the optimized geometrical structures of BCP-C₆₀, BCP-m1-C₆₀, and BCP-m2-C60 complexes, showing the shortest carbon-nitrogen distance between the BCP molecule and C₆₀. Referring to this, the shortest carbon-nitrogen distances of BCP-m1-C₆₀ and BCP-m2-C₆₀ were shorter than those of BCP-C₆₀, indicating that structural modification affects the interfacial interaction. That is, the increased planarity of BCP-m1 and BCP-m2 compared to BCP can contribute to a more compact molecular structure, thereby enhancing stability. Additionally, the nitrogen atoms on the control BCP were significantly exposed to air, while the nitrogen atoms on BCP-m1 and BCP-m2 were significantly protected by the bulky phenyl and tolyl groups, respectively. Moreover, when comparing BCP-m1 and BCP-m2, BCP-m2 containing tolyl groups was in a more protected environment. Consequently, BCP-m2 can suppress the reactivity of phenanthroline most effectively, enabling more stable operation within the device. This implies that introducing a phenyl group substituted with an alkyl or halogen functional group to the 2- and 9-positions of phenanthroline can reduce steric hindrance and increase molecular planarity, ultimately suppressing the reactivity of phenanthroline.

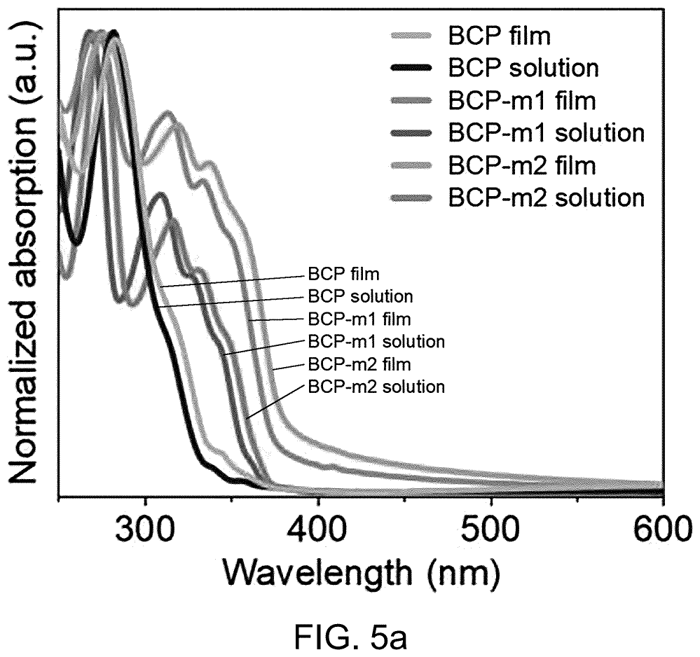

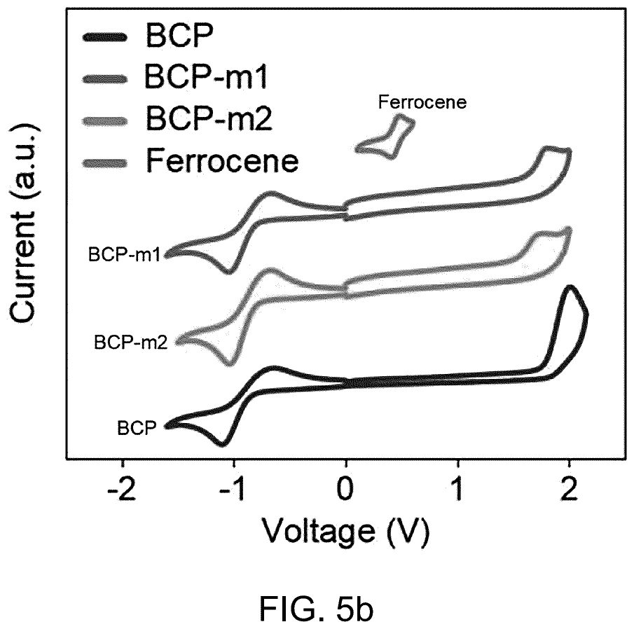

FIGS. 5a and 5b illustrate absorption spectra in solution and film states. Referring to this, the maximum absorption peak appeared at 282 nm for BCP, at 268 nm and 309 nm for BCP-m1, and at 275 nm and 316 nm for BCP-m2 in solution. That is, only BCP derivatives, BCP-m1 and BCP-m2, showed distinct shoulder absorption, which strongly suggests that BCP-m1 and BCP-m2 exhibit efficient π-conjugation between a BCP core and phenyl and tolyl terminal units, respectively. Additionally, when comparing BCP-m1 and BCP-m2, the absorbance of BCP-m2 was slightly red-shifted compared to BCP-m1. This is believed to be because the tolyl group on BCP-m2 donates more electrons than the phenyl group on BCP-m1, which enhances the 71-conjugation with the electron-withdrawing BCP core. In the film state, the control BCP showed a slight red shift in absorption, but there was little change in the overall absorption. This indicates that there was no efficient π-π stacking among the BCP molecules. On the contrary, the maximum absorption peaks were red-shifted to 272 nm and 314 nm for BCP-m1 and to 277 nm and 319 nm for BCP-m2. Additionally, the shoulder absorption was more intense and broader than that in the solution state, indicating that the intermolecular interactions between BCP-m1 and BCP-m2 are significantly enhanced in the film state.

As illustrated in FIG. 3, the phenyl ring on the control BCP was highly twisted from the BCP core unit, while BCP-m1 and BCP-m2 exhibited a fairly planar backbone structure. Thus, the absorption behavior in FIGS. 5a and 5b implies that BCP-m1 and BCP-m2 can have efficient π-conjugation and π-π intermolecular stacking due to the improvement in molecular planarity.

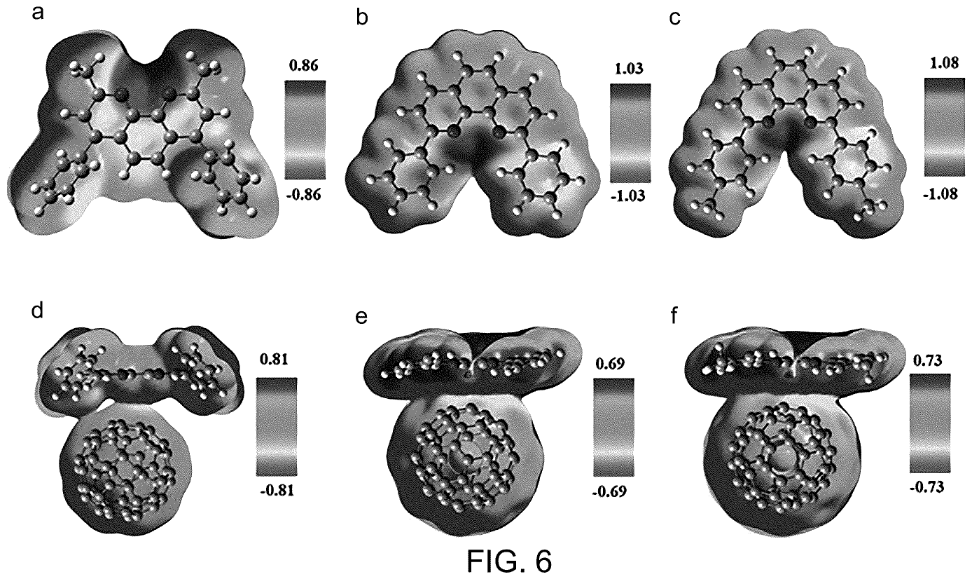

FIG. 6 illustrates (a-c) simulated electrostatic potential (ESP) maps of BCP, BCP-m1, and BCP-m2, and (d-f) simulated ESP maps of interactions between C₆₀ and BCP, BCP-m1, and BCP-m2. Electrostatic potential values, represented by a color scale, are expressed in electron volts (eV). Referring to this, the electrostatic potential (ESP) surfaces shown in a to c of FIG. 6 exhibited a trend similar to the optical analysis results. The enhanced π-electron delocalization within BCP-m1 and BCP-m2 was reflected in the ESP maps, suggesting an enhanced potential for intermolecular interactions. As illustrated in d to f of FIG. 6, the efficiently enhanced 71-interaction function and geometrically stable structure show that the BCP derivatives, BCP-m1 and BCP-m2 synthesized in the example, were energetically more stable than conventional BCP when deposited on C₆₀.

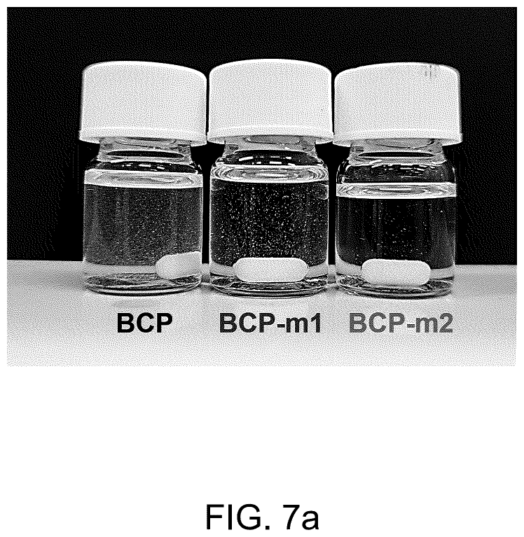

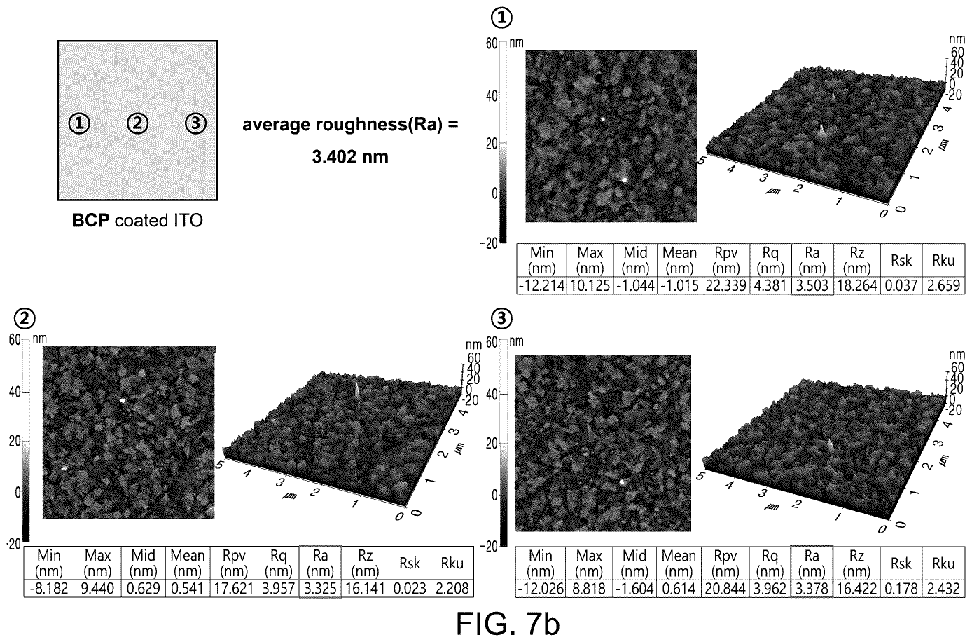

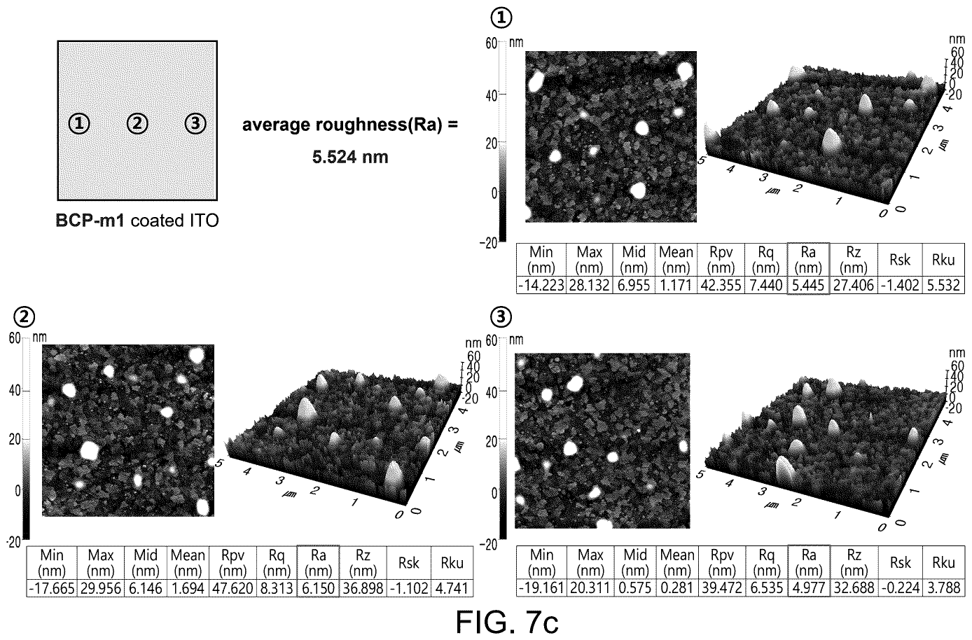

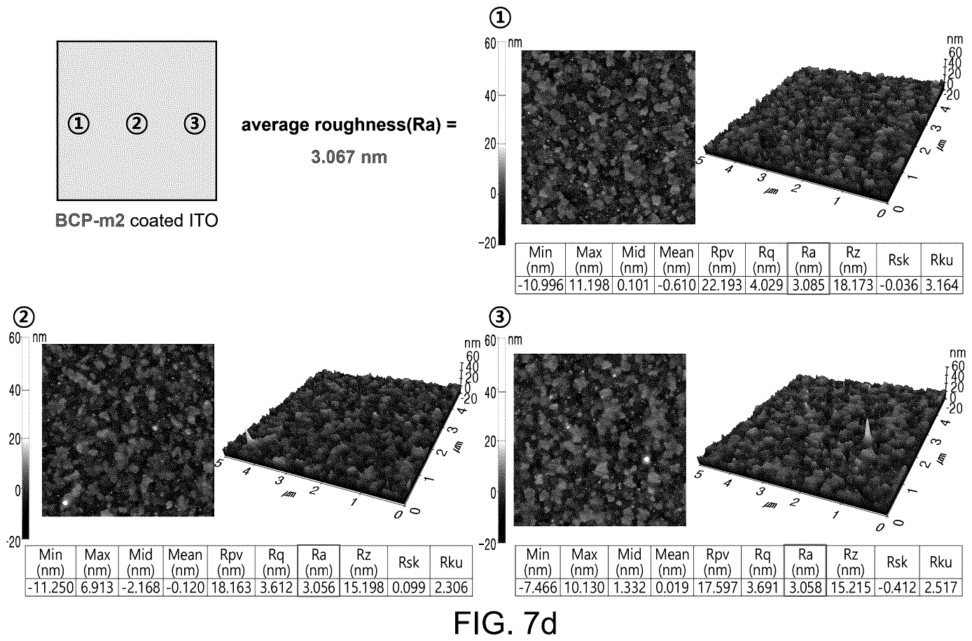

FIGS. 7a to 7d illustrate preparation of solutions of BCP, BCP-m1, and BCP-m2 for application in solution processes, and surface roughness (AFM) measurement results of thin films. Specifically, solutions were prepared by dissolving each derivative in IPA at a concentration of 0.5 mg/ml, followed by stirring at room temperature for 1 hour. The solubility was in the order of BCP<BCP-m1<BCP-m2. Subsequently, the BCP and BCP-m1 solutions were fully dissolved by stirring at 60° C. to 70° C. Each solution was spin-coated on an ITO glass substrate using a solution process and annealed at 70° C. for 5 minutes to produce thin-film samples. The uniformity of the prepared thin-film samples was measured using atomic force microscopy (AFM) equipment. As can be seen in FIGS. 7b to 7d, the average roughnesses (Ra) of the BCP, BCP-m1, and BCP-m2 thin films were 3.402 nm, 5.524 nm, and 3.067 nm, respectively. This implies that BCP-m2 is not only easy to dissolve, but also can produce a more uniform thin film through the same solution process. That is, for application in the solution process, it is considered advantageous for a functionalized phenyl group.

### <Experimental Example> Manufacture of Solar Cell

During a device fabrication process, substrates composed of etched indium tin oxide (ITO)-coated glass (1.1 mm thick, 10 Ω/sq, AMG) were thoroughly cleaned in an ultrasonic bath for 15 minutes using a 2% commercial detergent (Hellmanex) solution, deionized water, and ethanol, and then dried on a hot plate at 120° C. Subsequently, the substrates were treated with O₃/UV for 15 minutes and then spin-coated with a 200 μl solution of Meo-4PACz in methanol (0.36 mg/ml) at 2,000 rpm for 30 seconds with 2-second acceleration. The coated substrates were annealed at 100° C. for 10 minutes in a dry-air glove box with a relative humidity of equal to or less than 10%. Cs_{0.15}FA_{0.85}Pb(I_{0.95}Br_{0.05})₃ perovskite precursor solution (1.50 M), FAI (219.2 mg), PbI₂ (656.9 mg), PbBr₂ (27.5 mg), and CsI (58.4 mg) were mixed in 1 mL anhydrous dimethylformamide/dimethylsulfoxide (4:1, v:v) solvent and stirred at room temperature until fully dissolved. Immediately before use, oleylamine (0.004 M) was added to the perovskite precursor solution. The solution was spin-coated at 6,000 rpm for 60 seconds, and 400 mL of ethyl acetate (EA) was dropped onto the spinning substrates 40 seconds before the end of the process. To promote perovskite crystal formation, the substrates were sequentially heated at 100° C. for 30 minutes. After that, a 15 nm C₆₀ layer was deposited by thermal evaporation at a rate of 0.2 to 0.4 Å/s, and a 3 nm BCP layer was deposited at a rate of 0.1 Å/s to form a control group. For Examples 1 and 2, deposition was performed under same deposition conditions by replacing BCP with BCP-m1 and BCP-m2. Finally, a 100 nm silver electrode was deposited by thermal evaporation at a rate of 0.8 Å/s. The manufacturing process is illustrated in FIG. 8.

The PSC characteristics based on functional group modification within BCP were analyzed, and the results are illustrated in FIGS. 9 to 14.

First, FIG. 9 illustrates AFM images of (a) BCP, (c) BCP-m1, and (e) BCP-m2, and line profile graphs obtained from the AFM images of (b) BCP, (d) BCP-m1, and (f) BCP-m2. Referring to this, the BCP and BCP-m2 samples had rougher surfaces compared to the BCP-m1 sample. By line profiles extracted from the AFM images, this difference in roughness became more evident. BCP showed the highest roughness with an average Ra of 12.268 nm, followed by BCP-m2 with an Ra of 6.129 nm, and lastly BCP-m1 had the smoothest surface with an Ra of 3.120 nm.

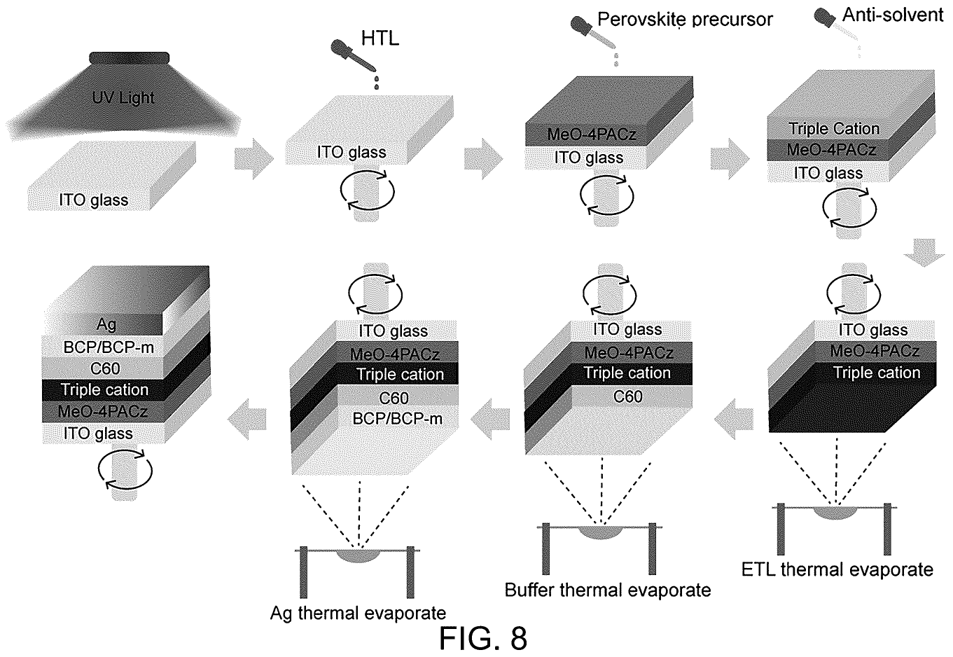

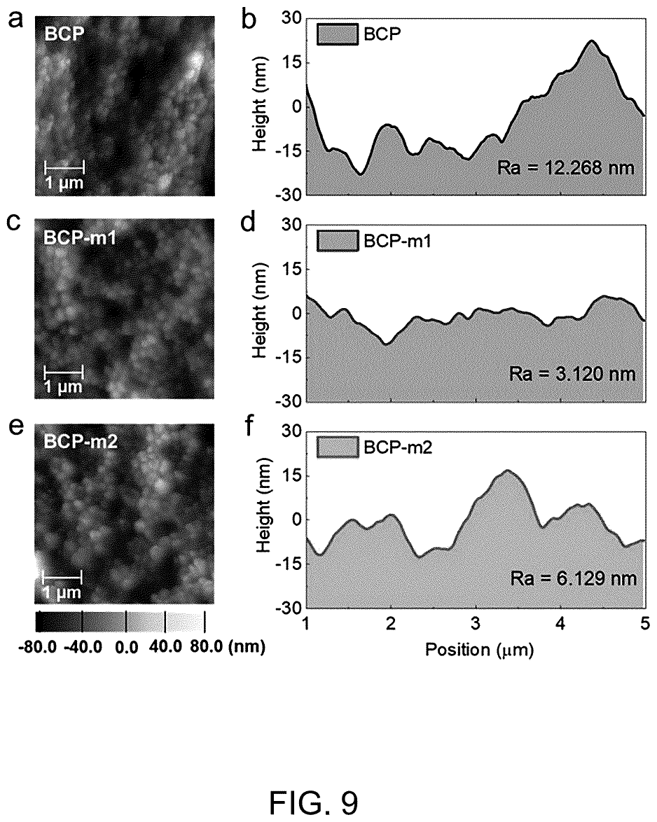

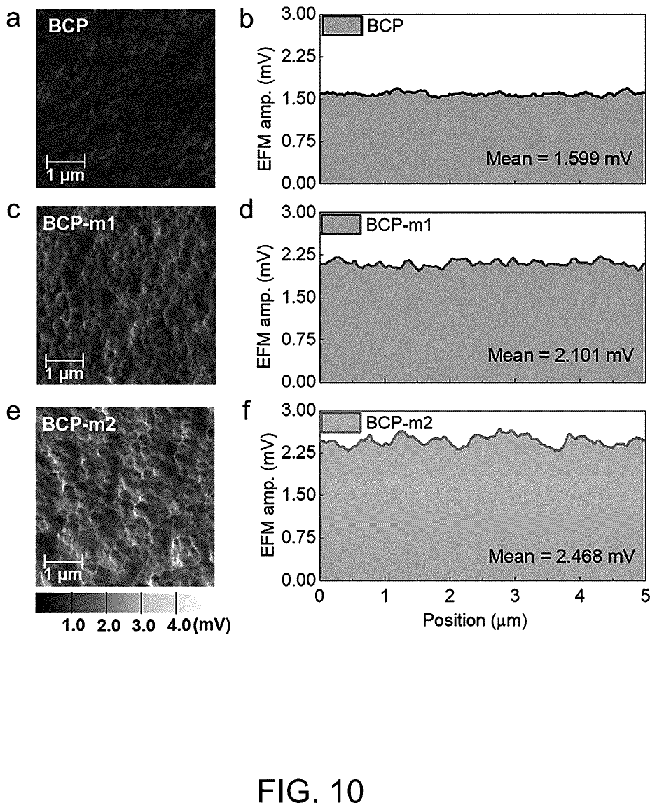

FIG. 10 illustrates EFM surface electrostatic force images of (a) BCP, (c) BCP-m1, and (e) BCP-m2, and line profile graphs obtained from the EFM images of (b) BCP, (d) BCP-m1, and (f) BCP-m2. Using EFM, a type of dynamic non-contact AFM, the surface electrostatic forces of the materials were investigated. Similar to the AFM analysis, line profiles were extracted from the EFM images to further assess the difference in surface electrostatic forces across the materials. Referring to a, c, and e of FIG. 10, the BCP, BCP-m1, and BCP-m2 samples exhibited distinct differences in the electrostatic forces. That is, the EFM image of the BCP sample showed dark brown color indicating the lowest electrostatic force, while that of the BCP-m1 sample showed a lighter brown color indicating a higher electrostatic force, and that of the BCP-m2 sample showed a bright yellow color indicating the highest electrostatic force among the samples. Also, referring to b, d, and f of FIG. 10, the electrostatic force on the surface of BCP was relatively evenly distributed, with a mean value of 1.599 mV. However, BCP-m1 and BCP-m2 exhibited less even distribution, with higher mean values of 2.101 mV and 2.468 mV, respectively. This is attributed to the conductivity of the materials. During EFM measurements, a DC current was applied to both the material surface and a cantilever tip. Therefore, in materials with higher conductivity, the interaction between the material and the cantilever tip was more pronounced, resulting in stronger color contrast in the EFM image and an increase in the surface electrostatic force. That is, the stronger color contrast and surface electrostatic force observed in BCP-m1 and BCP-m2 indicate enhanced conductivity compared to the original BCP.

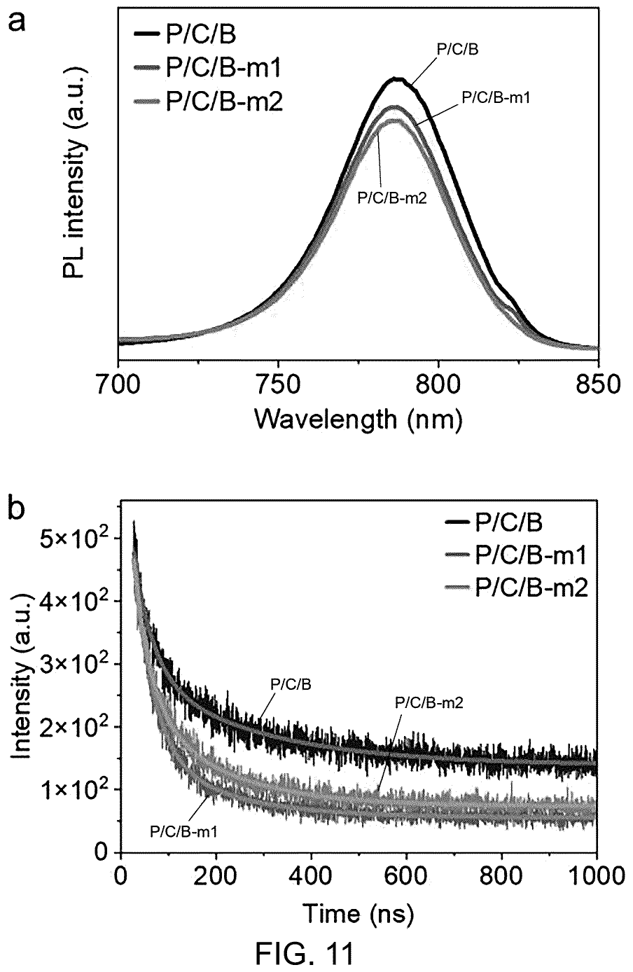

FIG. 11 illustrates graphs of (a) steady-state photoluminescence (PL) spectra and (b) time-resolved photoluminescence (TRPL) measurement results of perovskite with and without a top coating layer (where P represents perovskite, C represents C₆₀, and B represents BCP). Referring to a of FIG. 11, the steady-state PL results reveal that the PL intensity decreased when the BCP layer was replaced with BCP-m1 or BCP-m2. A lower PL intensity indicates a decrease in recombination of electron-hole pairs, implying that more electrons were successfully extracted from a PVSK layer. That is, as illustrated in FIG. 10, the surface electrostatic forces of BCP-m1 and BCP-m2 were found to be higher than that of the conventional BCP. Accordingly, the results in a of FIG. 11 reveal that the charge separation capability of BCP-m1 and BCP-m2 was enhanced compared to the conventional BCP due to the increase in the surface electrostatic force.

Additionally, referring to b of FIG. 11, the TRPL results were consistent with the steady-state PL results. The BCP-m1 and BCP-m2 samples exhibited faster photoluminescence decay compared to the BCP sample, indicating efficient charge extraction rather than fast recombination. This excellent charge extraction capability of BCP-m1 and BCP-m2 can reduce charge pair recombination, contributing to the enhanced PCE of the device. These results are consistent with the outcomes of prior studies, which demonstrate that faster TRPL decay is associated with enhanced charge extraction efficiency in perovskite-based devices.

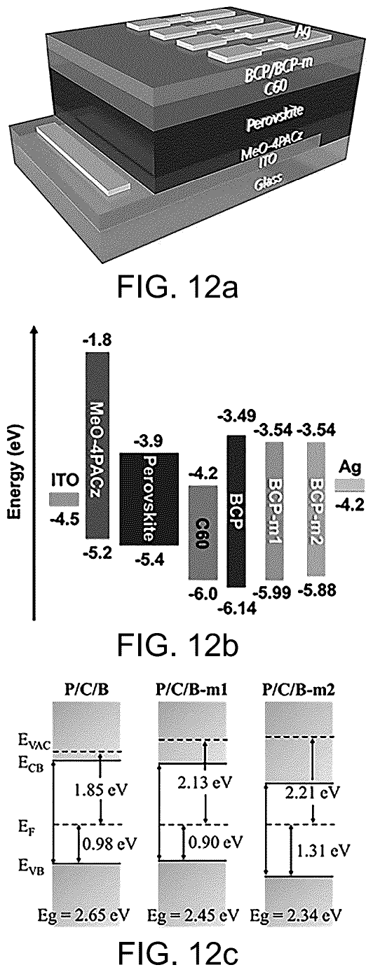

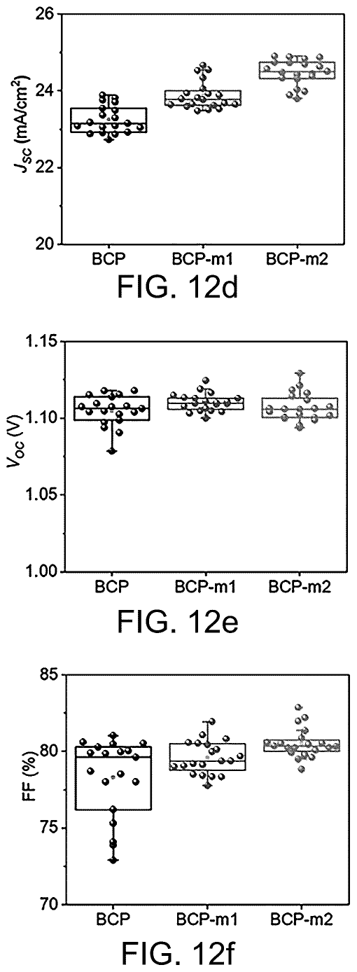

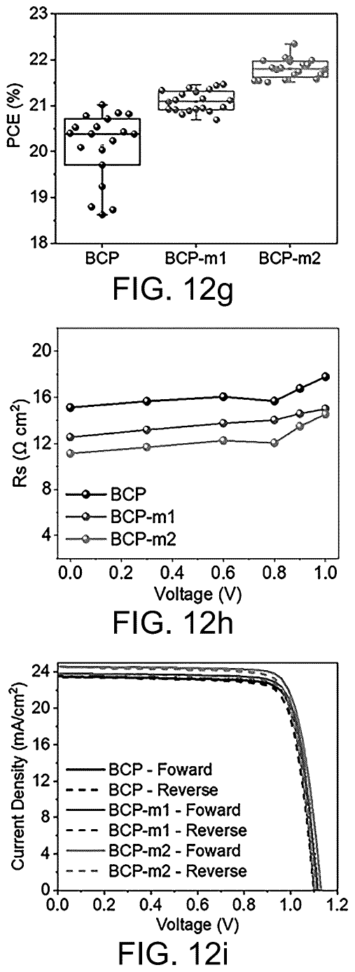

FIGS. 12a to 12i illustrate (a) a schematic structure of a perovskite solar cell, (b) a schematic diagram of bandgap alignment of BCPs, (c) a schematic band diagram of BCP, BCP-m1, and BCP-m2, a box chart showing statistical distribution of (d) current density (J_{SC}), (e) open-circuit voltage (V_{OC}), (f) fill factor (FF), and (g) power conversion efficiency (PCE) under simulated AM 1.5G sunlight, (h) a series resistance impedance spectroscopy graph of PSCs with BCP, BCP-m1, and BCP-m2, and (i) a graph of J-V curves in reverse scans (solid line) and forward scans (dash line) for perovskite solar cells under various conditions.

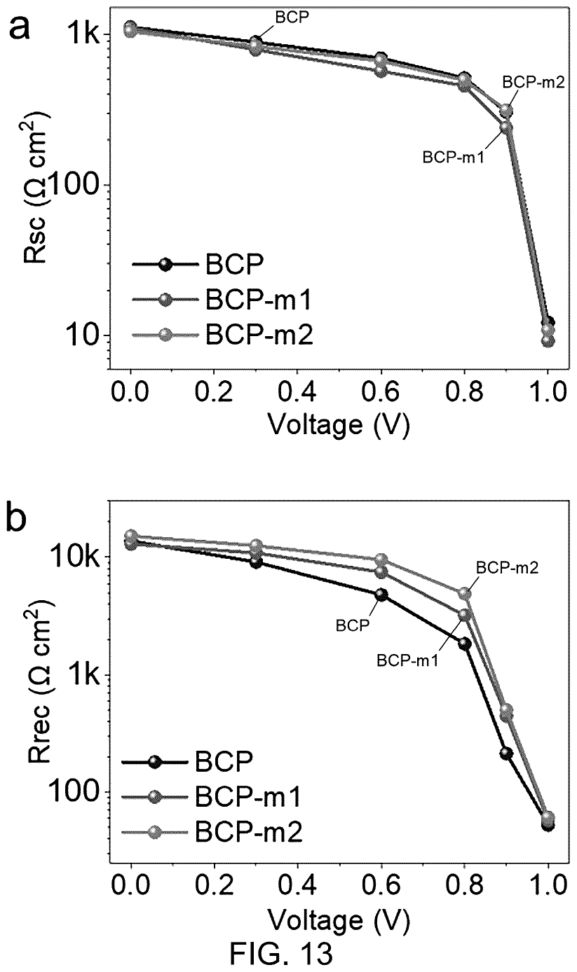

Referring to FIG. 12a, a device having a simple structure of ITO/MeO-4PACz/triple cation perovskite/C₆₀/BCP or BCP-m/Ag was fabricated using BCP, BCP-m1, and BCP-m2, and the band alignment diagram illustrated in b of FIG. 13 was constructed using cyclic voltammetry calculations. A slight change in the band gap was observed when BCP was modified. The bandgaps of BCP-m1 and BCP-m2 were smaller than that of BCP, and the smallest bandgap was for BCP-m2 having the same HOMO level. This reduction brought the LUMO level of BCP-m1 and BCP-m2 closer to the LUMO of C₆₀, which positively influenced electron injection from C₆₀ to an Ag electrode.

Additionally, Table 1 below illustrates current density (J_{SC}), open-circuit voltage (V_{OC}), (f) fill factor (FF), and (g) power conversion efficiency (PCE) values in FIGS. 12a to 12g.

TABLE 1

| Sample | | J_{sc} [mA cm⁻²] | V_{oc}[V] | FF[%] | PCE[%] |
|---|---|---|---|---|---|
| BCP | Maximum | 23.54 | 1.11 | 80.04 | 21.01 |
| | Average | 23.25 | 1.10 | 78.31 | 20.02 |
| BCP-m1 | Maximum | 23.78 | 1.11 | 81.06 | 21.46 |
| | Average | 23.88 | 1.11 | 79.58 | 21.10 |
| BCP-m2 | Maximum | 24.56 | 1.12 | 80.53 | 22.34 |
| | Average | 24.47 | 1.11 | 80.52 | 21.81 |

In this regard, referring to Table 1 and FIG. 13, the BCP-m2 sample significantly improved all major parameters (V_{OC} (open-circuit voltage), J_{SC} (short-circuit current density), FF (fill factor), and PCE) compared to the original BCP sample. In particular, the average PCE increased from 20.02% with a maximum efficiency of 21.01% for BCP to 22.34% with a maximum efficiency of 21.81% for BCP-m2. BCP-m1 also showed a slight improvement in efficiency compared to BCP, but remained lower than that achieved with BCP-m2. That is, this shows that the band alignment between material layers in PSCs plays a crucial role in understanding carrier conduction and separation.

In PSCs, resistance plays a significant role in determining the PCE of the device. The series resistance (Rs) represents the total resistive opposition that charge carriers (electrons and holes) face as they pass through different layers of the solar cell. Therefore, a lower series resistance leads to better charge transfer between layers, resulting in a higher PCE. Better charge transfer between layers also leads to a higher PCE. In this regard, referring to FIG. 12h, the series resistance of BCP-m1 and BCP-m2 was found to be lower than that of the control BCP. These results are consistent with the EFM results in FIG. 10, indicating that the higher surface electrostatic force of BCP-m1 and BCP-m2 corresponds to better conductivity.

Additionally, referring to Table 1 and FIG. 12i, BCP-m1 and BCP-m2 exhibited a clear improvement in hysteresis compared to the control BCP. That is, for hysteresis analysis, the J-V curve was scanned in both forward and reverse directions. The forward and reverse J-V curves for BCP-m1 or BCP-m2 showed minimal variations, implying that these modifications effectively reduced ion migration within the device. This reduced hysteresis indicates the enhanced stability and performance consistency of PSCs incorporating the BCP-m1 or BCP-m2 layer.

FIG. 13 illustrates graphs of (a) selective contact resistance and (b) recombination resistance by impedance spectroscopy of a PSC using BCP, BCP-m1, and BCP-m2. Referring to a of FIG. 13, there was no significant difference between the original BCP and the modified BCP in terms of selective contact resistance (Rsc). In PSCs, Rsc occurs at the interfaces where ETL and HTL selectively collect charge carriers. These results are consistent with the role of BCP as a buffer layer, suggesting that it predominantly conducts charges from the ETL to an electrode and thus exerts a minimal effect on charge collection. Also, referring to b of FIG. 13, BCP-m2 was found to have the highest recombination resistance among the samples. This recombination resistance refers to the resistance against recombination of charge carriers (electrons and holes) within the solar cell. A higher recombination resistance corresponds to a lower recombination rate, which is beneficial for achieving higher PCE in solar cells. Therefore, these results are consistent with the PL and TRPL results in FIG. 11, implying that BCP-m2 has a strong potential to suppress electron recombination, thereby improving the PCE of the device.

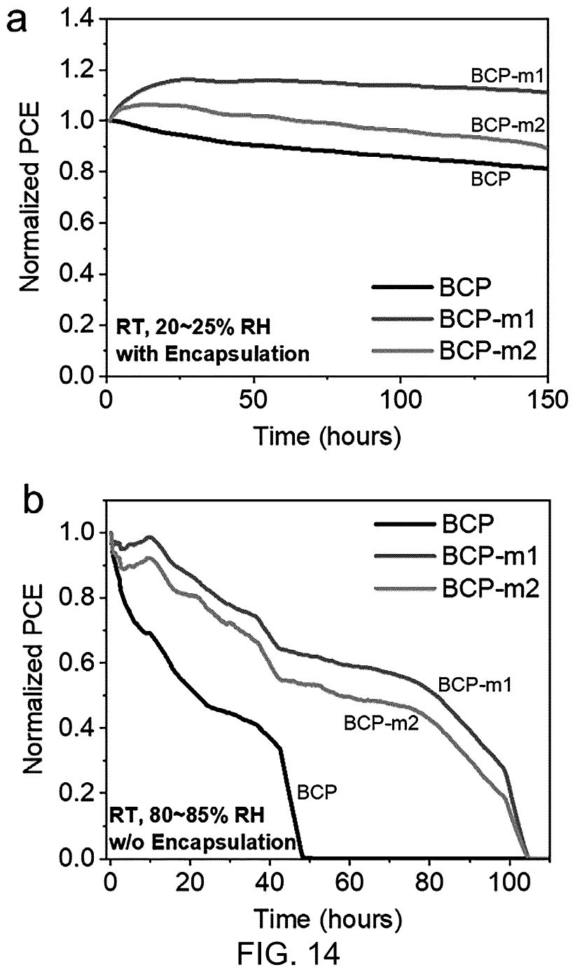

FIG. 14 illustrates MPPT graphs of a perovskite solar cell under light soaking (AM 1.5 G, 100 Mw cm⁻²) (a) in 20 to 25% humidity at room temperature with encapsulation and (b) in 80 to 85% humidity at room temperature without encapsulation. Specifically, to investigate the impact of the chemically stable structure where the reactive nitrogen atoms are embedded within the BCP-m molecules on the long-term stability of PSC devices, two different evaluation conditions were established. First, to isolate the effect of structural differences on long-term stability, the devices were encapsulated to prevent the interaction between the buffer layer molecules and water. Additionally, to assess whether the molecules designed for moisture resistance could maintain its stability under operating conditions, the maximum power point tracking (MPPT) was tracked without encapsulation in 80 to 85% relative humidity.

Referring to a of FIG. 14, the encapsulated BCP-m1 and BCP-m2 samples exhibited better long-term stability compared to the BCP sample. After 150 hours of measurement, the BCP sample exhibited the greatest decrease in PCE, dropping to about 80%, while the PCE of the BCP-m1 and BCP-m2 samples remained above 90%, with the BCP-m1 sample showing the highest stability. Also, referring to FIF. 14b, the control BCP sample exhibited a clear performance degradation compared to BCP-m1 and BCP-m2. The BCP sample failed to maintain performance for even 50 hours under high humidity, while both BCP-m1 and BCP-m2 maintained stable performance for over 100 hours, which is double the lifespan of the control BCP sample from the prior art. These long-term stability test results indicate that the structurally stable and tailor-made BCP-m molecules provide protection against moisture, thereby effectively enhancing the long-term stability of solar cells in high-humidity environments.

Based on the results of the above examples, it was demonstrated that the BCP derivative prepared in the present disclosure can significantly enhance the PCE and durability of solar cells by strategically modifying the conventional BCP functional group.

According to the present disclosure, by substituting methyl groups at the 2- and 9-positions of BCP with a phenyl group or a functionalized phenyl group and remaining the 4- and 7-positions of the phenanthroline core of BCP unsubstituted, the planarity and π-conjugation of the backbone can be significantly enhanced. In particular, substituting with a functionalized phenyl group allows the reactive nitrogen atoms of the phenanthroline core to be most effectively protected while maintaining a planar backbone structure. This enhances charge transport of PSCs and improves the device stability.

Therefore, the optoelectronic device including the novel BCP derivative of the present disclosure as a buffer layer can provide enhanced charge transfer compared to a perovskite solar cell having the conventional BCP as a buffer layer, resulting in enhanced device performance and contributing to greater device stability, reduced hysteresis, and enhanced long-term stability.

The foregoing has outlined rather broadly the features and technical advantages of the present disclosure so that those skilled in the art can better understand the claims that follow. It will be understood by those skilled in the art that the disclosure can be implemented in other specific forms without changing the spirit or essential features of the disclosure. Therefore, it should be noted that the forgoing embodiments are merely illustrative in all aspects and are not to be construed as limiting the disclosure. The scope of the disclosure is defined by the appended claims rather than the detailed description of the disclosure. All changes or modifications or their equivalents made within the meanings and scope of the claims should be construed as falling within the scope of the disclosure.

What is claimed is:

1. A bathocuproine (BCP) derivative, wherein a 1,10-phenanthroline compound is unsubstituted at 4- and 7-positions, and is symmetrically substituted at 2- and 9-positions with phenyl groups, alkylphenyl groups, halophenyl groups thereby providing improved molecular planarity.

2. The BCP derivative of claim 1, wherein the derivative has Chemical Formula 1 below: 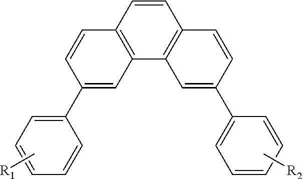
(where R₁, R₂ are each independently selected from the group consisting of hydrogen, an alkyl group having 1 to 4 carbon atoms, and a halogen atom).

3. An optoelectronic device, wherein a first electrode, a hole transport layer, a photoactive layer, an electron transport layer, and a second electrode are sequentially stacked, and
wherein the photoactive layer is a light-absorbing layer or a light-emitting layer,
an electron transport buffer layer containing the BCP derivative of claim 1 is further provided between the electron transport layer and the second electrode.

4. A perovskite solar cell, wherein a first electrode, a hole transport layer, a photoactive layer, an electron transport layer, and a second electrode are sequentially stacked, and
wherein the photoactive layer is formed to include a perovskite compound represented by the chemical formula APbX₃ or ASnX₃ (where A is a metal cation, an organic cation, or a combination thereof, and X is selected from at least one of I, Br, and Cl),
the electron transport layer is formed to include a fullerene compound, and
an electron transport buffer layer containing the BCP derivative of claim 1 is further provided between the electron transport layer and the second electrode.

5. The perovskite solar cell of claim 4, wherein the first electrode includes at least one selected from the group consisting of a flexible transparent electrode substrate, indium tin oxide (ITO), fluorine tin oxide (FTO), aluminum zinc oxide (AZO), boron-doped zinc oxide (BZO), niobium titanium oxide (NTO), zinc tin oxide (ZTO), and indium zinc tin oxide (IZTO), and
the second electrode includes at least one selected from the group consisting of silver (Ag), aluminum (Al), platinum (Pt), tungsten (W), copper (Cu), molybdenum (Mo), gold (Au), nickel (Ni), palladium (Pd), and carbon (C).

6. The perovskite solar cell of claim 4, wherein the perovskite solar cell has a power conversion efficiency of at least 90% after 10 hours of operation under room temperature and low humidity conditions (20 to 25% RH).

7. The perovskite solar cell of claim 4, wherein the perovskite solar cell has a power conversion efficiency of at least 50% after 50 hours of operation under room temperature and high humidity conditions (80 to 85% RH).

8. A method of manufacturing a perovskite solar cell, the method including:
forming a first electrode;
forming a hole transport layer on the first electrode;
forming a photoactive layer by coating a perovskite compound of APbX₃ or ASnX₃ (where A is a metal cation, an organic cation, or a combination thereof, and X is at least one selected from I, Br, and Cl) on the hole transport layer, followed by heat treatment;
forming an electron transport layer by depositing a fullerene compound on the photoactive layer;
forming a buffer layer by depositing the BCP derivative of claim 1 on the electron transport layer; and
forming a second electrode on the buffer layer.
